(19) United States
(12) Patent Application Publication
(10) Pub. No.: US 2026/0176654 A1
(43) Pub. Date: Jun. 25, 2026

(54) METHOD OF INTRODUCING A SUBSTANCE INTO A CELL, GENETICALLY MODIFIED CELL AND METHOD OF PRODUCING THE SAME, AND CONTROL PROGRAM FOR APPARATUS FOR INTRODUCING A SUBSTANCE INTO A CELL

(71) Applicants: Yokogawa Electric Corporation, Tokyo (JP); NATIONAL UNIVERSITY CORPORATION TOKYO UNIVERSITY OF AGRICULTURE AND TECHNOLOGY, Tokyo (JP)

(72) Inventors: Makoto Mochiduki, Tokyo (JP); Daisuke Nojima, Allschwil (CH); Tsuneji Sawai, Tokyo (JP); Tsuyoshi Tanaka, Tokyo (JP); Tomoko Yoshino, Tokyo (JP); Kaoruko Akasaka, Tokyo (JP)

(73) Assignees: Yokogawa Electric Corporation, Tokyo (JP); NATIONAL UNIVERSITY CORPORATION TOKYO UNIVERSITY OF AGRICULTURE AND TECHNOLOGY, Tokyo (JP)

(21) Appl. No.: 19/426,368

(22) Filed: Dec. 19, 2025

(30) Foreign Application Priority Data
Dec. 24, 2024 (JP) 2024-227927
Nov. 14, 2025 (JP) 2025-195592

(51) Int. Cl. C12N 15/89; C12N 5/00; C12N 5/10

(52) U.S. Cl. CPC C12N 15/89; C12N 5/0062; C12N 5/10; C12N 2510/00; C12N 2513/00

(57) ABSTRACT

A method of introducing a substance into a cell includes a) seeding floating cells on a gel plate medium having a specific hardness, b) filling the gel plate medium seeded with cells with an electrolytic solution and positioning a nanopipette filled with a substance at a cell-corresponding position in the electrolytic solution, c) measuring a current between an inside of the nanopipette and the electrolytic solution and moving the nanopipette in a cell direction to a position at which a drop rate from a steady state current is a set current drop rate, d) moving the nanopipette in the cell direction by a set penetration distance to puncture a cell, e) applying a voltage between the inside of the nanopipette and the electrolytic solution to eject the substance into the cell, and f) removing the nanopipette.

A presentation material “Delivery of biomaterials into single-microalgal cells using a nanopipette” was presented at Molecular Life of Diatom Japan on Jan. 12, 2024. The author(s) of the “Delivery of biomaterials into single-microalgal cells using a nanopipette” is (are) Rein Yasui, Kaoruko Akasaka, Tomoko Yoshino, Daisuke Nojima, Makoto Mochiduki, Tsuyoshi Tanaka who is (are) the inventor(s) of the present application. A copy of the presentation material is provided on a concurrently filed Information Disclosure Statement pursuant to the guidance of 78 Fed. Reg. 11076 (Feb. 14, 2013).

A presentation material “Consideration of the Conditions for Introducing a Substance into Microalgae Using a Nanopipette” was presented at The 24th annual meeting of the Japanese Society for Marine Biotechnology on May 27, 2024. The author(s) of the “Consideration of the Conditions for Introducing a Substance into Microalgae Using a Nanopipette” is (are) Kaoruko Akasaka, Rein Yasui, Tomoko Yoshino, Daisuke Nojima, Makoto Mochiduki, Tsuyoshi Tanaka who is (are) the inventor(s) of the present application. A copy of the presentation material is provided on a concurrently filed Information Disclosure Statement pursuant to the guidance of 78 Fed. Reg. 11076 (Feb. 14, 2013).

A presentation material “Microinjection System for Single-Microalgal Cells Based on Electroosmotic Flow” was presented at PRIME 2024 on Oct. 6-11, 2024. The author(s) of the “Microinjection System for Single-Microalgal Cells Based on Electroosmotic Flow” is (are) Kaoruko Akasaka, Rein Yasui, Tomoko Yoshino, Daisuke Nojima, Makoto Mochiduki, Tsuyoshi Tanaka who is (are) the inventor(s) of the present application. A copy of the presentation material is provided on a concurrently filed Information Disclosure Statement pursuant to the guidance of 78 Fed. Reg. 11076 (Feb. 14, 2013).

### CROSS-REFERENCE TO RELATED APPLICATION

The present application claims priorities to Japanese Patent Application 2024-227927 filed on Dec. 24, 2024 and Japanese Patent Application 2025-195592 filed on Nov. 14, 2025, the entire contents of which are incorporated herein by reference.

### TECHNICAL FIELD

The present disclosure relates to a method of introducing a substance into a cell, a genetically modified cell and a method of producing the same, and a control program for an apparatus for introducing a substance into a cell.

### BACKGROUND

Until now, the establishment of strains of cells, particularly cells having a cell wall, that have properties suitable for use as hosts for substance production has had to rely on inefficient and rigid methods, such as the spontaneous generation of strains with specific traits or induction by treatment with mutagens. Against this background, the introduction of substances such as genome editing tools and nucleic acids in a cell has been considered as a more efficient method for targeted gene modification. The electroporation method described in Patent Literature (PTL) 1 and 2 and the nanopipette method described in PTL 3, for example, have been considered as methods that enable the introduction of substances into a cell.

In the electroporation method, the introduction of a substance into a cell having a cell wall is usually easily hindered by the presence of the cell wall, making severe pulse conditions required to increase the efficiency of substance introduction. However, since severe pulse conditions are likely to damage cells, it is often difficult to achieve both low damage to cells and high introduction efficiency with the electroporation method.

For example, in the electroporation method described in PTL 1, if the number of electroporation pulses is increased in order to increase the introduction efficiency, particularly in the case of cells having a cell wall such as microalgae, the survival rate of the cells decreases. If the applied voltage is increased to improve the introduction efficiency, then the cell survival rate decreases and cells stick together, depending on the applied voltage. Furthermore, not only the cells but also the substances to be introduced into the cells (e.g., nucleic acids, proteins, and other substances) are damaged, which may make the introduction of the substances itself difficult. Also, in the case of cells that contain a large number of ions, such as cells of marine species, sparks are likely to occur when a pulse is applied, making it difficult to introduce substances in a minimally invasive manner.

The electroporation method described in PTL 2 weakens the property of inhibiting substance introduction by isolating microalgae with missing outer shells, which are extremely rare in nature, and performing electroporation treatment on the microalgae with missing outer shells. However, damage to cells during the electroporation process and the resulting risk of unexpected cell behavior still tend to occur. In addition, it is necessary to isolate a strain lacking the outer shell (cell wall), which requires many complicated processes. Strains lacking an outer shell have low environmental resistance and are prone to cell death due to, for example, UV light, temperature changes, and physical stimuli. Although it is possible to obtain microalgae lacking an outer shell by performing an enzymatic method using cellulase to remove cell walls, this technique cannot be applied to cell walls other than cellulose (such as diatoms). Furthermore, in the case of species that secrete large amounts of extracellular polysaccharides, enzymatic treatment becomes difficult. It is also possible to obtain microalgae that lack an outer shell through genetic engineering, but this requires complicated procedures.

The nanopipette method described in PTL 3 has not been applied to penetrating a floating cell having a cell wall. In particular, in cells having a cell wall, such as microalgae, the cell wall is hard, and when the tip of the pipette touches a cell, the cell moves and cannot be penetrated.

### CITATION LIST

### Patent Literature

PTL 1: JP 2004-33070 A
PTL 2: JP 2015-181402 A
PTL 3: WO2013/012452

### SUMMARY

We predicted that puncturing with a nanopipette that has a tip diameter of several tens of nanometers and ejecting a substance using ionic voltage would enable both high substance introduction efficiency into cells and high cell survival rate. By seeding cells onto a gel plate medium of a specific hardness and introducing the substance using a nanopipette, we then succeeded in achieving substance introduction with both high substance introduction efficiency and high cell survival rate. This technique makes it possible to introduce substances such as nucleic acids and genome editing tools into cells, thereby contributing to improved breeding efficiency.

One aspect of the present disclosure is as follows.

A method of introducing a substance into a cell, the method comprising:
a) seeding floating cells on a gel plate medium having a hardness such that, by a hardness evaluation according to JIS K6503, a breaking load is 2.0 N to 30 N, Young's modulus is 0.5×10⁵ Pa to 7×10⁵ Pa, or 1 mm pressing stress is 1.0 N to 8 N;
b) filling the gel plate medium seeded with cells with an electrolytic solution and positioning a nanopipette filled with a substance at a cell-corresponding position in the electrolytic solution;
c) measuring a current between an inside of the nanopipette and the electrolytic solution and moving the nanopipette in a cell direction to a position at which a drop rate from a steady state current is a set current drop rate of 2% or more and 50% or less;
d) moving the nanopipette in the cell direction by a set penetration distance of 1 μm or more and 50 μm or less to penetrate a cell;
e) applying a voltage between the inside of the nanopipette and the electrolytic solution to eject the substance into the cell; and
f) retracting the nanopipette.

One aspect of the present disclosure is as follows.

A method of introducing a substance into a cell, the method comprising:
a′)
   a′-1) seeding floating cells onto a substrate and layering a gel on the cells;
   a′-2) peeling the gel from the substrate to obtain a gel sheet with the cells transferred to a gel surface;
   a′-3) layering the gel sheet on a gel plate medium so that a cell transfer surface faces up, and setting a hardness of layered gel overall, by a hardness evaluation according to JIS K6503, to a breaking load of 2.0 N to 30 N, Young's modulus of 0.5×10⁵ Pa to 7×10⁵ Pa, or 1 mm pressing stress of 1.0 N to 8 N;
b) filling the gel plate medium seeded with cells with an electrolytic solution and positioning a nanopipette filled with a substance at a cell-corresponding position in the electrolytic solution;
c) measuring a current between an inside of the nanopipette and the electrolytic solution and moving the nanopipette in a cell direction to a position at which a drop rate from a steady state current is a set current drop rate of 2% or more and 50% or less;
d) moving the nanopipette in the cell direction by a set penetration distance of 1 μm or more and 50 μm or less to penetrate a cell;
e) applying a voltage between the inside of the nanopipette and the electrolytic solution to eject the substance into the cell; and
f) retracting the nanopipette.

One aspect of the present disclosure is as follows.

A method of producing a genetically modified cell, the method comprising:
a) seeding floating cells on a gel plate medium having a hardness such that, by a hardness evaluation according to JIS K6503, a breaking load is 2.0 N to 30 N, Young's modulus is 0.5×10⁵ Pa to 7×10⁵ Pa, or 1 mm pressing stress is 1.0 N to 8 N;
b) filling the gel plate medium seeded with cells with an electrolytic solution and positioning a nanopipette filled with a substance at a cell-corresponding position in the electrolytic solution;
c) measuring a current between an inside of the nanopipette and the electrolytic solution and moving the nanopipette in a cell direction to a position at which a drop rate from a steady state current is a set current drop rate of 2% or more and 50% or less;
d) moving the nanopipette in the cell direction by a set penetration distance of 1 μm or more and 50 μm or less to penetrate a cell;
e) applying a voltage between the inside of the nanopipette and the electrolytic solution to eject the substance into the cell; and
f) retracting the nanopipette.

One aspect of the present disclosure is as follows.

A method of producing a genetically modified cell, the method comprising:
a′)
   a′-1) seeding floating cells onto a substrate and layering a gel on the cells;
   a′-2) peeling the gel from the substrate to obtain a gel sheet with the cells transferred to a gel surface;
   a′-3) layering the gel sheet on a gel plate medium so that a cell transfer surface faces up, and setting a hardness of layered gel overall, by a hardness evaluation according to JIS K6503, to a breaking load of 2.0 N to 30 N, Young's modulus of 0.5×10⁵ Pa to 7×10⁵ Pa, or 1 mm pressing stress of 1.0 N to 8 N;
b) filling the gel plate medium seeded with cells with an electrolytic solution and positioning a nanopipette filled with a substance at a cell-corresponding position in the electrolytic solution;
c) measuring a current between an inside of the nanopipette and the electrolytic solution and moving the nanopipette in a cell direction to a position at which a drop rate from a steady state current is a set current drop rate of 2% or more and 50% or less;
d) moving the nanopipette in the cell direction by a set penetration distance of 1 μm or more and 50 μm or less to penetrate a cell;
e) applying a voltage between the inside of the nanopipette and the electrolytic solution to eject the substance into the cell; and
f) retracting the nanopipette.

One aspect of the present disclosure is as follows.

A genetically modified cell produced by a method comprising:
a) seeding floating cells on a gel plate medium having a hardness such that, by a hardness evaluation according to JIS K6503, a breaking load is 2.0 N to 30 N, Young's modulus is 0.5×10⁵ Pa to 7×10⁵ Pa, or 1 mm pressing stress is 1.0 N to 8 N;
b) filling the gel plate medium seeded with cells with an electrolytic solution and positioning a nanopipette filled with a substance at a cell-corresponding position in the electrolytic solution;
c) measuring a current between an inside of the nanopipette and the electrolytic solution and moving the nanopipette in a cell direction to a position at which a drop rate from a steady state current is a set current drop rate of 2% or more and 50% or less;
d) moving the nanopipette in the cell direction by a set penetration distance of 1 μm or more and 50 μm or less to penetrate a cell;
e) applying a voltage between the inside of the nanopipette and the electrolytic solution to eject the substance into the cell; and
f) retracting the nanopipette.

One aspect of the present disclosure is as follows.

A genetically modified cell produced by a method comprising:
a′)
   a′-1) seeding floating cells onto a substrate and layering a gel on the cells;
   a′-2) peeling the gel from the substrate to obtain a gel sheet with the cells transferred to a gel surface;
   a′-3) layering the gel sheet on a gel plate medium so that a cell transfer surface faces up, and setting a hardness of layered gel overall, by a hardness evaluation according to JIS K6503, to a breaking load of 2.0 N to 30 N, Young's modulus of 0.5×10⁵ Pa to 7×10⁵ Pa, or 1 mm pressing stress of 1.0 N to 8 N;
b) filling the gel plate medium seeded with cells with an electrolytic solution and positioning a nanopipette filled with a substance at a cell-corresponding position in the electrolytic solution;
c) measuring a current between an inside of the nanopipette and the electrolytic solution and moving the nanopipette in a cell direction to a position at which a drop rate from a steady state current is a set current drop rate of 2% or more and 50% or less;
d) moving the nanopipette in the cell direction by a set penetration distance of 1 μm or more and 50 μm or less to penetrate a cell;
e) applying a voltage between the inside of the nanopipette and the electrolytic solution to eject the substance into the cell; and
f) retracting the nanopipette.

One aspect of the present disclosure is as follows.

A control program for an apparatus for introducing a substance into a cell, the control program comprising instructions for executing processing comprising:
a) positioning a nanopipette filled with a substance at a cell-corresponding position in an electrolytic solution in a gel plate medium of a specific hardness, the gel plate medium being seeded with floating cells and filled with the electrolytic solution;
b) measuring a current between an inside of the nanopipette and the electrolytic solution and moving the nanopipette in a cell direction to a position at which a drop rate from a steady state current is a set current drop rate of 2% or more and 50% or less;
c) moving the nanopipette in the cell direction by a set penetration distance of 1 μm or more and 50 μm or less to penetrate a cell;
d) applying a voltage between the inside of the nanopipette and the electrolytic solution to eject the substance into the cell; and
e) retracting the nanopipette.

### Advantageous Effect

According to the present disclosure, it is possible to provide a method of introducing a substance into a cell, the method achieving both high substance introduction efficiency and a high cell survival rate. Furthermore, using this method of introducing a substance, the present disclosure can provide an automatically controlled system for introducing a substance into a cell. This technique makes it possible to introduce substances such as nucleic acids and genome editing tools into cells, thereby contributing to improved breeding efficiency.

### BRIEF DESCRIPTION OF THE DRAWINGS

In the accompanying drawings:

FIG. 1 is a schematic diagram of a system for use with the method of the present disclosure and a pipette approach step in the method of the present disclosure;

FIG. 2 is a conceptual diagram of a pipette penetration step in the method of the present disclosure;

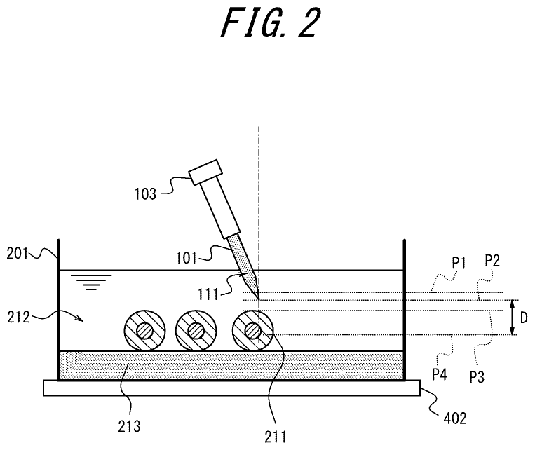

FIG. 3 is a schematic diagram of a pipette substance ejection step in the method of the present disclosure;

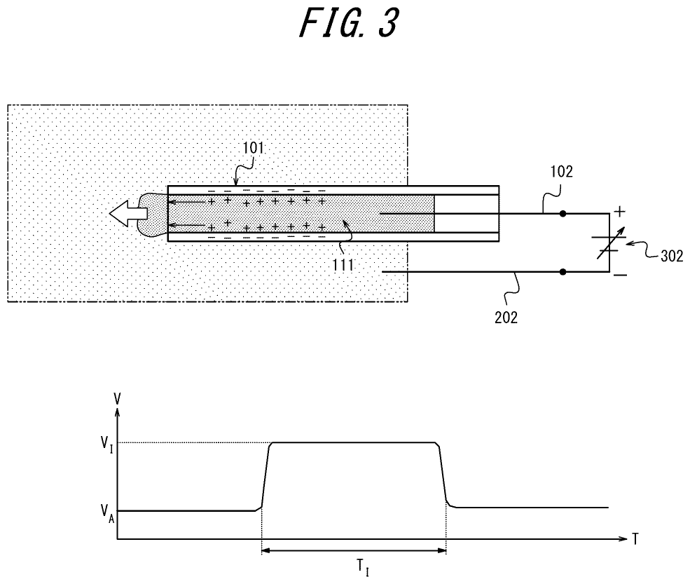

FIG. 4 is a diagram illustrating an example control mechanism of a system for use with the method of the present disclosure;

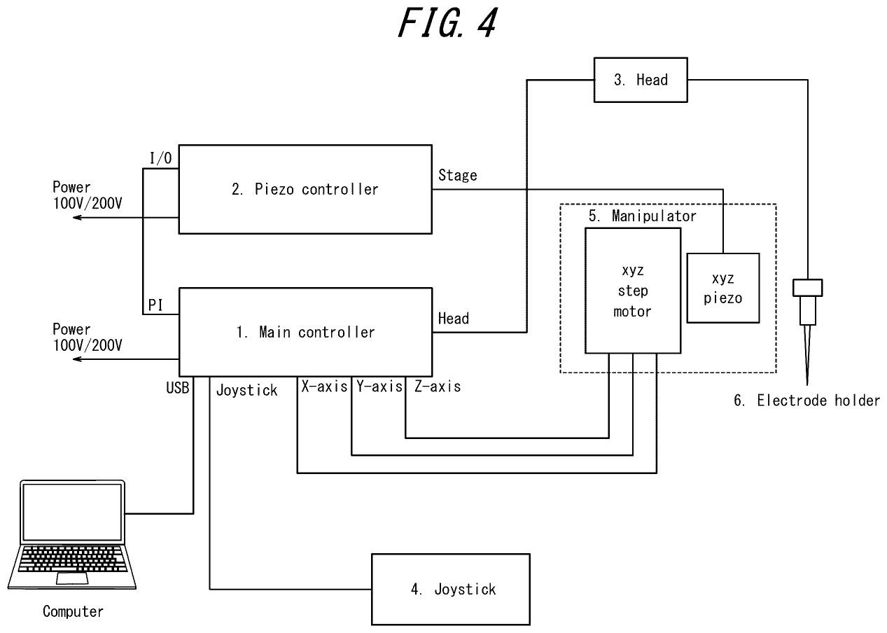

FIG. 5 is a schematic diagram of the overall scheme of the method of the present disclosure for seeding cells in a gel plate medium; and

FIG. 6 is a schematic diagram of the overall scheme of the method of the present disclosure for transferring cells to a gel sheet.

### DETAILED DESCRIPTION

Demand exists for a method to increase the efficiency of introducing a substance into a floating cell, in particular for a method to increase the efficiency of introducing a substance into a floating cell having a cell wall. Substance production using the metabolism of microorganisms such as microalgae, filamentous fungi, Escherichia coli, and yeast is being widely examined as a method that can produce compounds that are difficult to synthesize chemically while reducing the environmental impact. Among these microorganisms, microalgae are expected to be hosts for producing substances through photosynthesis. However, when attempting to breed microalgae through genetic modification, it has been difficult to achieve both high efficiency in introducing protein complexes, such as nucleic acids and genome editing tools, into cells and a high cell survival rate due in particular to the presence of the microalgae cell wall.

It would be helpful to provide a method of introducing a substance into a cell, the method achieving both high substance introduction efficiency and a high cell survival rate. Furthermore, it would be helpful to use this method of introducing a substance to provide an automatically controlled system for introducing a substance into a cell.

An outline of the present disclosure is as follows.

[1] A method of introducing a substance into a cell, the method comprising:
a) seeding floating cells on a gel plate medium having a hardness such that, by a hardness evaluation according to JIS K6503, a breaking load is 2.0 N to 30 N, Young's modulus is 0.5×10⁵ Pa to 7×10⁵ Pa, or 1 mm pressing stress is 1.0 N to 8 N;
b) filling the gel plate medium seeded with cells with an electrolytic solution and positioning a nanopipette filled with a substance at a cell-corresponding position in the electrolytic solution;
c) measuring a current between an inside of the nanopipette and the electrolytic solution and moving the nanopipette in a cell direction to a position at which a drop rate from a steady state current is a set current drop rate of 2% or more and 50% or less;
d) moving the nanopipette in the cell direction by a set penetration distance of 1 μm or more and 50 μm or less to penetrate a cell;
e) applying a voltage between the inside of the nanopipette and the electrolytic solution to eject the substance into the cell; and
   f) retracting the nanopipette.

[2] A method of introducing a substance into a cell, the method comprising:
a′)
   a′-1) seeding floating cells onto a substrate and layering a gel on the cells;
   a′-2) peeling the gel from the substrate to obtain a gel sheet with the cells transferred to a gel surface;
   a′-3) layering the gel sheet on a gel plate medium so that a cell transfer surface faces up, and setting a hardness of layered gel overall, by a hardness evaluation according to JIS K6503, to a breaking load of 2.0 N to 30 N, Young's modulus of 0.5×10⁵ Pa to 7×10⁵ Pa, or 1 mm pressing stress of 1.0 N to 8 N;
b) filling the gel plate medium seeded with cells with an electrolytic solution and positioning a nanopipette filled with a substance at a cell-corresponding position in the electrolytic solution;
c) measuring a current between an inside of the nanopipette and the electrolytic solution and moving the nanopipette in a cell direction to a position at which a drop rate from a steady state current is a set current drop rate of 2% or more and 50% or less;
d) moving the nanopipette in the cell direction by a set penetration distance of 1 μm or more and 50 μm or less to penetrate a cell;
e) applying a voltage between the inside of the nanopipette and the electrolytic solution to eject the substance into the cell; and
f) retracting the nanopipette.

[3] The method according to [1] or [2], wherein the substance is a protein, a mixture or complex containing a protein, a mixture or complex containing a protein and a nucleic acid, a nucleic acid, or a dye.

[4] The method according to [3], wherein the substance is a substance for genome editing.

[5] The method according to [3], wherein
the substance is a positively charged substance or a negatively charged substance,
in a case in which the substance is a positively charged substance, e) is performed by applying a voltage so that the inside of the nanopipette is at a positive potential and the electrolytic solution is at a negative potential, and
in a case in which the substance is a negatively charged substance, e) is performed by applying a voltage so that the inside of the nanopipette is at a negative potential and the electrolytic solution is at a positive potential.

[6] The method according to [5], wherein e) is performed by applying a voltage at a set applied voltage of −11 V or more and +11 V or less and a set application time of 0.1 seconds or more and 5.0 seconds or less.

[7] A method of producing a genetically modified cell, the method comprising:
a) seeding floating cells on a gel plate medium having a hardness such that, by a hardness evaluation according to JIS K6503, a breaking load is 2.0 N to 30 N, Young's modulus is 0.5×10⁵ Pa to 7×10⁵ Pa, or 1 mm pressing stress is 1.0 N to 8 N;
b) filling the gel plate medium seeded with cells with an electrolytic solution and positioning a nanopipette filled with a substance at a cell-corresponding position in the electrolytic solution;
c) measuring a current between an inside of the nanopipette and the electrolytic solution and moving the nanopipette in a cell direction to a position at which a drop rate from a steady state current is a set current drop rate of 2% or more and 50% or less;
d) moving the nanopipette in the cell direction by a set penetration distance of 1 μm or more and 50 μm or less to penetrate a cell;
e) applying a voltage between the inside of the nanopipette and the electrolytic solution to eject the substance into the cell; and
f) retracting the nanopipette.

[8] A method of producing a genetically modified cell, the method comprising:
a′)
   a′-1) seeding floating cells onto a substrate and layering a gel on the cells;
   a′-2) peeling the gel from the substrate to obtain a gel sheet with the cells transferred to a gel surface;
   a′-3) layering the gel sheet on a gel plate medium so that a cell transfer surface faces up, and setting a hardness of layered gel overall, by a hardness evaluation according to JIS K6503, to a breaking load of 2.0 N to 30 N, Young's modulus of 0.5×10⁵ Pa to 7×10⁵ Pa, or 1 mm pressing stress of 1.0 N to 8 N;
b) filling the gel plate medium seeded with cells with an electrolytic solution and positioning a nanopipette filled with a substance at a cell-corresponding position in the electrolytic solution;
c) measuring a current between an inside of the nanopipette and the electrolytic solution and moving the nanopipette in a cell direction to a position at which a drop rate from a steady state current is a set current drop rate of 2% or more and 50% or less;
d) moving the nanopipette in the cell direction by a set penetration distance of 1 μm or more and 50 μm or less to penetrate a cell;
e) applying a voltage between the inside of the nanopipette and the electrolytic solution to eject the substance into the cell; and
f) retracting the nanopipette.

[9] A genetically modified cell produced by a method comprising:
a) seeding floating cells on a gel plate medium having a hardness such that, by a hardness evaluation according to JIS K6503, a breaking load is 2.0 N to 30 N, Young's modulus is 0.5×10⁵ Pa to 7×10⁵ Pa, or 1 mm pressing stress is 1.0 N to 8 N;
b) filling the gel plate medium seeded with cells with an electrolytic solution and positioning a nanopipette filled with a substance at a cell-corresponding position in the electrolytic solution;
c) measuring a current between an inside of the nanopipette and the electrolytic solution and moving the nanopipette in a cell direction to a position at which a drop rate from a steady state current is a set current drop rate of 2% or more and 50% or less;
d) moving the nanopipette in the cell direction by a set penetration distance of 1 μm or more and 50 μm or less to penetrate a cell;
e) applying a voltage between the inside of the nanopipette and the electrolytic solution to eject the substance into the cell; and
f) retracting the nanopipette.

[10] A genetically modified cell produced by a method comprising:
a′)
   a′-1) seeding floating cells onto a substrate and layering a gel on the cells;
   a′-2) peeling the gel from the substrate to obtain a gel sheet with the cells transferred to a gel surface;
   a′-3) layering the gel sheet on a gel plate medium so that a cell transfer surface faces up, and setting a hardness of layered gel overall, by a hardness evaluation according to JIS K6503, to a breaking load of 2.0 N to 30 N, Young's modulus of 0.5×10⁵ Pa to 7×10⁵ Pa, or 1 mm pressing stress of 1.0 N to 8 N;
b) filling the gel plate medium seeded with cells with an electrolytic solution and positioning a nanopipette filled with a substance at a cell-corresponding position in the electrolytic solution;
c) measuring a current between an inside of the nanopipette and the electrolytic solution and moving the nanopipette in a cell direction to a position at which a drop rate from a steady state current is a set current drop rate of 2% or more and 50% or less;
d) moving the nanopipette in the cell direction by a set penetration distance of 1 μm or more and 50 μm or less to penetrate a cell;
e) applying a voltage between the inside of the nanopipette and the electrolytic solution to eject the substance into the cell; and
f) retracting the nanopipette.

[11] A control program for an apparatus for introducing a substance into a cell, the control program comprising instructions for executing processing comprising:
a) positioning a nanopipette filled with a substance at a cell-corresponding position in an electrolytic solution in a gel plate medium of a specific hardness, the gel plate medium being seeded with floating cells and filled with the electrolytic solution;
b) measuring a current between an inside of the nanopipette and the electrolytic solution and moving the nanopipette in a cell direction to a position at which a drop rate from a steady state current is a set current drop rate of 2% or more and 50% or less;
c) moving the nanopipette in the cell direction by a set penetration distance of 1 μm or more and 50 μm or less to penetrate a cell;
d) applying a voltage between the inside of the nanopipette and the electrolytic solution to eject the substance into the cell; and
e) retracting the nanopipette.

Hereinafter, the present disclosure will be described in detail with reference to the drawings as necessary. However, the drawings are merely examples for explaining the present disclosure, and the technical scope of the present disclosure is not limited by the examples in the drawings.

[System for Introducing Substance into a Cell]

A system (system for introducing a substance into a cell, hereinafter referred to as the “system of the present disclosure”) for use in the methods of the present disclosure (method for introducing a substance into a cell and method of producing a genetically modified cell) is described. The system of the present disclosure includes, for example, the following components:
a nanopipette;
a three-dimensional (also referred to as “xyz” for convenience) movement pipette holder for moving the nanopipette in three dimensions (xyz);
an electrode capable of contacting a liquid filled inside the nanopipette (hereinafter referred to as the “pipette electrode”);
an electrode capable of contacting an electrolytic solution in the cell holder (hereinafter referred to as the “reference electrode”);
a current measurement circuit for measuring the current between the pipette electrode and the reference electrode; and
a voltage application circuit for applying a voltage between the pipette electrode and the reference electrode.

Additionally, the system of the present disclosure may further include a position input device for moving a three-dimensional movement module by manual input. The system of the present disclosure is installed on an optical microscope for use. In addition, the system of the present disclosure is used by placing the cell holder on the optical microscope, the cell holder holding cells, a gel plate medium, and an electrolytic solution in which the cells are immersed, and by holding, inside the nanopipette, a solution containing a substance to be introduced into the cells.

Each component will be described below.

### (Nanopipette)

The term “nanopipette” refers to a tubular structure having a nanoscale tip opening. A nanoscale tip opening is, for example, a conical tip opening (i.e., a nanopore) of about 10 nm to about 500 nm, preferably about 50 nm (+20%). The material used for the nanopipette is an inert, non-biological material, such as glass or quartz. The inner wall of the nanopipette may be surface-treated to suppress adsorption of substances (e.g., nucleic acids, proteins) filled inside the nanopipette. The nanopipette preferably has a shape or scale that allows an electrode to be inserted into the nanopipette so as to contact the solution within the nanopipette.

Nanopipettes have a single channel (also called a “barrel” or “bore”) or a plurality of parallel channels within a tube. A nanopipette that has a single channel within its tube is also called a “single-barreled nanopipette”. A nanopipette having a plurality of channels within its tube is also called a “multi-barreled nanopipette”. A nanopipette having two parallel channels within its tube is also called a “double-barreled nanopipette”. The nanopipette used in the method of the present disclosure is preferably a single-barreled nanopipette from the perspective of ease and reliability of the operation of filling the nanopipette with a substance, and accuracy of current measurement and voltage application.

Nanopipettes are commercially available (e.g., product number SU10ACC-NP01 manufactured by Yokogawa). Nanopipettes can also be made by, for example, drawing glass or quartz capillaries with a laser.

Details of the nanopipette are described, for example, in WO2014/160036 and WO2013/012452 (PTL 3).

### (Three-Dimensional (Xyz) Movement Pipette Holder)

The “three-dimensional (xyz) movement pipette holder” is a pipette holder to which a nanopipette is attached and which moves the attached nanopipette in three dimensions by driving a rough actuator and a fine actuator. The nanopipette is attached to the three-dimensional movement pipette holder so that two of the three directions (for convenience, also referred to as the “x-axis direction” and the “y-axis direction”) are perpendicular or nearly perpendicular to the long axis of the nanopipette, and the remaining direction (for convenience, also referred to as the “z-axis direction”) is parallel or nearly parallel to the long axis of the nanopipette. The three-dimensional movement pipette holder may be configured by, for example, a holder stage that is driven by the rough actuator and a holder head that is driven by the fine actuator, is mounted on the holder stage, and has the nanopipette attached thereto.

A “rough actuator” is a three-dimensional actuator that allows rough positioning, such as an actuator with a stroke on the order of 10 mm to 100 mm and a setting resolution on the order of 0.1 μm to 1 μm. Examples of rough actuators include electromagnetic force-driven actuators such as actuators based on motors (rotary motors, linear motors).

A “fine actuator” is a three-dimensional actuator that allows fine positioning, such a three-dimensional actuator with a stroke on the order of 100 μm to 500 μm and setting resolution on the order of 1 nm. Examples of fine actuators include piezoelectric effect driven actuators, such as piezoelectric element-based actuators. Furthermore, if the xy axis setting resolution of the rough actuator has sufficient performance for the target cell size, the fine actuator may be specialized for precise approach to and penetration of cells and be limited to one dimension, i.e., only the Z axis.

### (Position Input Device)

The position input device is a device for manually inputting and indicating the position of the nanopipette. Examples of the position input device include a pointing device such as a joystick, a key input device such as a keyboard, and a combination of these.

### (Pipette Electrode, Reference Electrode)

Examples of the pipette electrode and the reference electrode include a gold electrode, a silver electrode (such as a silver tetrakis(4-chlorophenyl) borate (AgTBACI) electrode or an Ag/AgCl electrode), a platinum electrode, and the like. The reference electrode may be used in contact with the electrolytic solution at the cell holder. Also, when the nanopipette is a multi-barreled (e.g., double-barreled) nanopipette, the pipette electrode may be placed in a flow path that holds the substance to be introduced into the cell, and the reference electrode may be placed in another flow path.

### (Current Measurement Circuit, Voltage Application Circuit)

The current measurement circuit is a circuit for measuring the ionic current between the pipette electrode and the reference electrode (i.e., the current between the inside of the nanopipette and the electrolytic solution). The current measurement circuit preferably has a current measurement range of the order of about 100 pA to 100 nA and is preferably capable of measuring a current change (reduction) of about 2% to 50% for cell surface detection. For example, in a case in which the current (steady state current) is 10 nA at a point sufficiently far from the cell surface, if detection of the cell surface is set as a 20% decrease in current, the point at which the current becomes 8 nA is measured. Other examples include a low-noise amplifier circuit for accurately detecting a very small steady state reference current and changes in the current. Low noise may also be achieved using software-based digital filtering techniques.

The current measured by the current measurement circuit is used as an indicator of the distance between the cell and the tip of the nanopipette according to the principles of the scanning ion conductance microscopy (SICM) technique. When the tip of the nanopipette comes into contact with the electrolytic solution in the cell holder, an ionic current begins to flow between the tip and the reference electrode. Next, even if the tip of the nanopipette is moved closer to the cell, there is no significant change in the current value if the tip is sufficiently far from the cell surface. The current value at this time is called the “steady state current”. Subsequently, upon the tip of the nanopipette coming extremely close to the cell, the current drops off rapidly according to the distance between the cell and the nanopipette tip. This is because the cell membrane is a highly insulating film. Therefore, by automatically controlling the nanopipette to temporarily stop when the drop rate of current from the steady state current reaches a preset value (hereinafter referred to as the “set current drop rate”), the nanopipette can be automatically paused, thereby automatically pausing the tip of the nanopipette immediately next to the cell.

### (Voltage Application Circuit)

The voltage application circuit is a circuit for applying a voltage between the pipette electrode and the reference electrode (between the inside of the nanopipette and the electrolytic solution). The voltage application circuit is preferably a circuit capable of applying a voltage on the order of −11 V to +11 V with time control on the order of 0.01 seconds.

### (Cell)

The method of the present disclosure is applicable particularly to cells having a cell wall, and particularly to cells that have a cell wall and can become floating cells (hereinafter also referred to as “floating cell having a cell wall”). Regardless of the above, the present disclosure is also applicable to cells that have no cell wall or cells that have partially lost their cell wall. Although microalgae are exemplified as floating cells, having a cell wall or having lost their cell wall, that are applicable to the present disclosure, any floating cells having a cell wall are applicable. Applicable types of cells include microorganisms such as microalgae cells, yeast cells, Escherichia coli, and filamentous fungal cells. Examples of microalgae include the genera Tetraselmis, Nannochloropsis, Dunaliella, Phaeodactylum, Isochrysis, Chlorella, Haematococcus, Spirulina, Scenedesmus, and Chlamydomonas. Examples of yeast include the genera Saccharomyces, Candida, Pichia, Kluyveromyces, Zygosaccharomyces, Schizosaccharomyces, Debaryomyces, Hansenula, Torulopsis, and Yarrowia. Examples of Escherichia coli include the genus Escherichia. Examples of filamentous fungi include the genera Aspergillus, Penicillium, Rhizopus, Fusarium, Trichoderma, Mucor, Neurospora, Alternaria, Beauveria, and Cladosporium. Cells that have no cell wall or have partially lost their cell wall and that are applicable to the method of the present disclosure include cells that originally lack cell walls, such as cells of the genus Euglena. Furthermore, cells having the cell wall described above can be genetically modified or subjected to protoplast treatment (decomposition and removal of the cell wall using enzymes or chemicals) to produce cells without a cell wall or cells that have partially lost their cell wall, and these cells can then be applied to the method of the present disclosure.

### (Cell Holder)

The cell holder for holding the cells, gel plate medium, and electrolytic solution is not particularly limited, but typically a transparent holder with an openable top is used. Examples of such holders include cell culture vessels such as cell culture dishes and multi-well plates, the top of which is covered with an openable lid, and flat plates such as glass slides.

### (Cell Immersion Electrolytic Solution)

Examples of the electrolytic solution in which the cells are immersed in the cell holder include common culture media for the culture of the cell type used, and buffered saline (e.g., phosphate buffered saline (PBS), HEPES buffered saline (HBS), Hank's balanced salt solution (HBSS), or the like).

As a medium for microalgae, appropriate media can be used alone or in combination depending on the type of strain used, but mainly depending on whether the target microalgae live in freshwater or saltwater. Examples of media for freshwater microalgae include BG11 medium, Z8 medium, ASM-11 medium, TAP medium, CHU-10 medium, WC medium, BBM medium, and AAP medium. Examples of media for marine microalgae include f/2 medium, ESM medium, and MNK medium.

Examples of media for Escherichia coli include LB medium, TSA medium, NA medium, MacConkey medium, EMB medium, M9 minimal medium, SOB medium, and TB medium, although the media vary depending on the type of strain used. These media may be used alone or in combination.

Examples of media for yeast include YPD medium, YPG medium, SD medium, SGal medium, PDA medium, SC medium, YMM medium, and YM medium, although the media vary depending on the type of strain used. These media may be used alone or in combination.

Examples of media for filamentous fungi include PDA medium, SDA medium, Czapek-Dox medium, MEA medium, CMA medium, V8 Juice Agar medium, YES medium, OA medium, Czapek yeast extract medium, and Emerson YpSs medium, although the media vary depending on the type of strain used. These media may be used alone or in combination.

### (Gel Plate Medium)

A gel plate medium is a medium that has been gelled to a particular hardness. A gel plate medium can be obtained, for example, from a mixture of a medium and a gelling agent. Gelling media are generally used for the selection of cell colonies, but there have been no reports of their use for optimizing injection into floating cells. In the present disclosure, by using a gel plate medium for injection into floating cells, it is possible to achieve the unexpected excellent effect of combining high substance introduction efficiency with high cell survival rate. Examples of the medium include those described above. Examples of gelling agents include agarose, agar, lambda-carrageenan, kappa-carrageenan, iota-carrageenan, gellan gum, xanthan gum, guar gum, gum arabic, Gelrite, pectin, methylcellulose, glucomannan, gelatin, collagen, corn starch, sodium alginate, and calcium alginate, and these can be added to the medium as gelling agents either alone or in combination.

The specific hardness of the gel plate medium is a hardness optimized for injection, using a nanopipette, in a floating cell. Breaking load, Young's modulus, or pressing stress, for example, can be used as an indicator of the hardness. The breaking load, Young's modulus, and pressing stress can be measured by a hardness evaluation according to JIS K6503. When the breaking load is used as an indicator, the specific hardness of the gel plate medium is a breaking load of 2.0 N or more and 30 N or less, preferably 2.2 N or more, more preferably 5 N or more, preferably 27 N or less, more preferably 20 N or less. When Young's modulus is used as an indicator, the specific hardness of the gel plate medium is a Young's modulus of 0.5×10⁵ Pa or more and 7×10⁵ Pa or less, preferably 0.75×10⁵ Pa or more, more preferably 2.5×10⁵ Pa or more, preferably 5.6×10⁵ Pa or less, and more preferably 5×10⁵ Pa or less. When the pressing stress is used as an indicator, the specific hardness of the gel plate medium is a 1 mm pressing stress of 1.0 N or more and 8 N or less, preferably 1.3 N or more, more preferably 3 N or more, preferably 7.2 N or less, more preferably 7 N or less. The hardness of the gel plate medium can be appropriately adjusted depending on the type and concentration of the gelling agent. The gel plate medium may be a single layer or a multi-layer in which the same or different gels are layered, so long as the hardness of the entire medium is within the above range. If it is difficult to stably evaluate the hardness due to reasons such as the thinness of the gel plate medium, a gel plate medium sample for hardness measurement, with a thickness of 2 mm to 10 mm, can be separately prepared with the same medium composition and the hardness measurement value can be applied.

(Substance Introduced into Cell)

The substance to be introduced into the cell is not particularly limited, but examples thereof include proteins, mixtures or complexes containing proteins, mixtures or complexes containing proteins and nucleic acids, nucleic acids, and dyes. The substance to be introduced into the cell is not particularly limited but is preferably a substance that dissolves or is suspended in an electrolytic solution. The substance is also preferably an electrically charged substance. The electrically charged substance may be a single charged substance, a mixture or complex of plurality of substances that is charged as a whole, or a substance (single substance or a mixture or complex of a plurality of substances) that, when dissolved or suspended in an electrolytic solution, causes the solution as a whole to be charged. Electrically charged substances include positively charged substances and negatively charged substances. The substance to be introduced into the cell (e.g., a protein, a mixture or complex containing a protein, a mixture or complex containing a protein and a nucleic acid, a nucleic acid, or a dye) is preferably either a positively charged substance or a negatively charged substance. For example, a protein, a mixture or complex of proteins, a mixture or complex containing nucleic acid and protein, or the like may be either a positively charged substance or a negatively charged substance. They may, for example, be a positively charged substance. An example of a mixture or complex containing a nucleic acid and a protein is a substance for genome editing. Substances for genome editing include CRISPR-Cas9, CRISPR-Cas3, ZFN, TALEN, and PPR systems. For example, nucleic acids (e.g., DNA, RNA, and the like) may be either a positively charged substance or a negatively charged substance. They may, for example, be a negatively charged substance. The substance introduced into the cell may be a marker substance such as a dye or may contain a marker substance.

The agent introduced into the cell is usually in the form of a liquid (e.g., a solution). When the substance to be introduced into the cell is in the form of a solution, the solvent may be an aqueous electrolytic solution or a non-aqueous electrolytic solution, with an aqueous electrolytic solution being preferred. Examples of the aqueous electrolytic solution include those exemplified above as the electrolytic solution into which the cells are immersed.

[Method of Introducing a Substance into a Cell]

In one embodiment, a method of introducing a substance into a cell of the present disclosure includes the following steps.
a) seeding floating cells on a gel plate medium of a specific hardness (floating cell seeding step);
b) filling the gel plate medium seeded with cells with an electrolytic solution and positioning a nanopipette filled with a substance at a cell-corresponding position in the electrolytic solution (pipette positioning step);
c) measuring a current between an inside of the nanopipette and the electrolytic solution and moving the nanopipette in a cell direction to a position at which a drop rate from a steady state current is a set current drop rate of 2% or more and 50% or less (pipette approach step);
d) moving the nanopipette in the cell direction by a set penetration distance of 1 μm or more and 50 μm or less to penetrate a cell (pipette penetration step);
e) applying a voltage between the inside of the nanopipette and the electrolytic solution to eject the substance into the cell (substance ejection step); and
f) retracting the nanopipette (pipette retraction step).

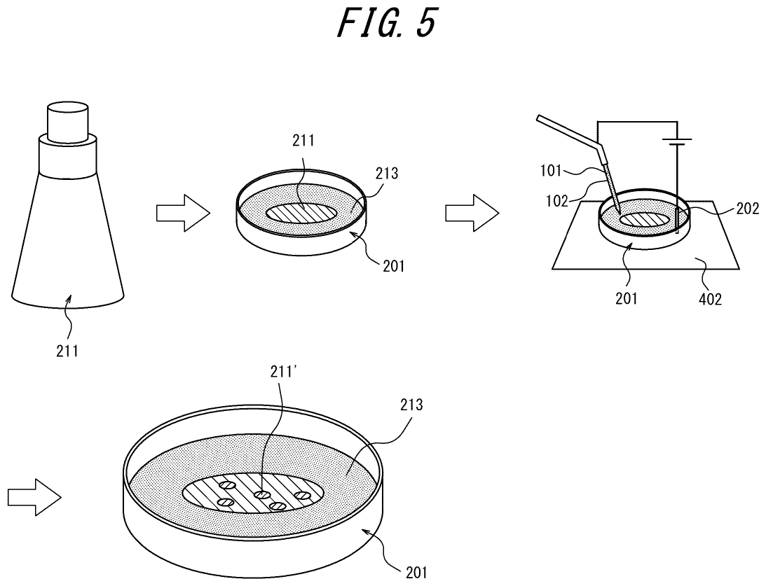

The overall scheme of the method of the present disclosure in the present embodiment is illustrated in FIG. 5. Floating cells (211) are cultured (first scheme in FIG. 5), the cultured floating cells (211) are seeded in a gel plate medium (213) in a cell holder (201) (second scheme in FIG. 5), a substance is introduced into the cells (211) using a nanopipette (101) (third scheme in FIG. 5), and a colony (211′) of cells into which the substance has been introduced is obtained (fourth scheme in FIG. 5). In the method of the present disclosure, when floating cells are fixed on a gel plate of a specific hardness and combined with specific injection conditions using a nanopipette, it is possible to achieve both high efficiency of substance introduction into floating cells and high cell survival rate. By fixing floating cells to a gel plate with a specific hardness, the cells can receive an injection without moving when the nanopipette comes into contact with the cells.

In another embodiment, a method of introducing a substance into a cell of the present disclosure includes the following steps.
a′) (floating cell gel sheet transfer step)
   a′-1) seeding floating cells onto a substrate and layering a gel on the cells;
   a′-2) peeling the gel from the substrate to obtain a gel sheet with the cells transferred to a gel surface;
   a′-3) layering the gel sheet on a gel plate medium so that a cell transfer surface faces up, and setting a hardness of layered gel overall, by a hardness evaluation according to JIS K6503, to a breaking load of 2.0 N to 30 N, Young's modulus of 0.5×10⁵ Pa to 7×10⁵ Pa, or 1 mm pressing stress of 1.0 N to 8 N;
b) filling the gel plate medium seeded with cells with an electrolytic solution and positioning a nanopipette filled with a substance at a cell-corresponding position in the electrolytic solution (pipette positioning step);
c) measuring a current between an inside of the nanopipette and the electrolytic solution and moving the nanopipette in a cell direction to a position at which a drop rate from a steady state current is a set current drop rate of 2% or more and 50% or less (pipette approach step);
d) moving the nanopipette in the cell direction by a set penetration distance of 1 μm or more and 50 μm or less to penetrate a cell (pipette penetration step);
e) applying a voltage between the inside of the nanopipette and the electrolytic solution to eject the substance into the cell (substance ejection step); and
f) retracting the nanopipette (pipette retraction step).

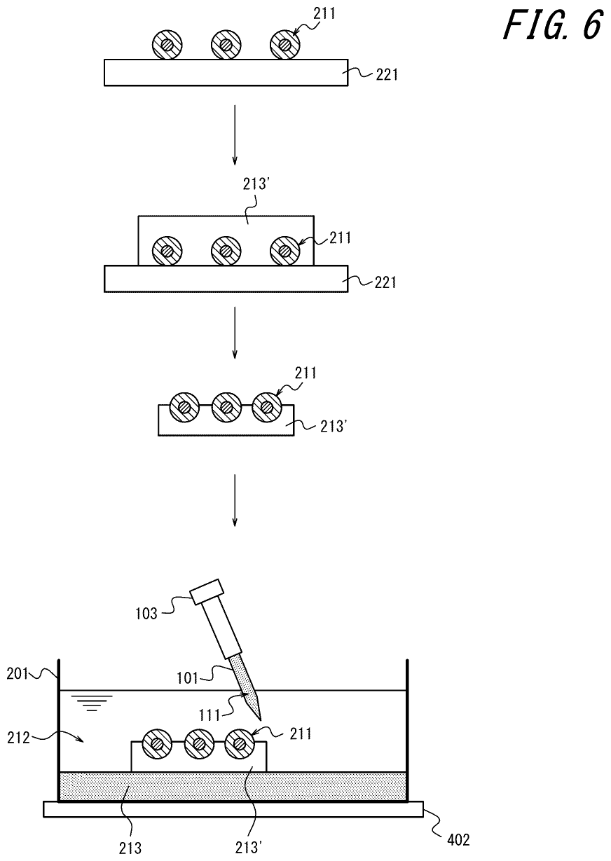

The overall scheme of the method of the present disclosure in the present embodiment is illustrated in FIG. 6. Cells (211) are seeded on a substrate (221) (first scheme in FIG. 6), a gel is layered on the cells (211) on the substrate (221) to form a gel sheet (213′) (second scheme in FIG. 6), the gel sheet (213′) is peeled off from the substrate (221) and the cells (211) are transferred to the gel sheet (213′) (third scheme in FIG. 6), the gel sheet (213′) to which the cells (211) have been transferred is layered on a gel plate medium (213), and a substance (111) is introduced into the cells (211) using a nanopipette (101) (fourth scheme in FIG. 6). The method of layering a gel sheet, which has cells transferred onto a gel, on a gel plate makes it easier to inhibit cell migration, thereby achieving a higher success rate of substance introduction.

### (Step a): Floating Cell Seeding Step)

Step a) is a step of seeding cells in the form of floating cells onto a gel plate medium. Cells can be made into floating cells by, for example, culturing them in a liquid medium. The seeding of floating cells may be carried out by, for example, seeding a culture solution containing floating cells onto a gel plate medium.

(Step a′): Floating Cell Gel Sheet Transfer Step)

Step a′) is a step of transferring cells in a state of floating cells onto a gel sheet and layering the gel sheet onto which the cells have been transferred on a gel plate medium. The substrate is not particularly limited, but examples thereof include slide glass, plastic plates, cell culture dishes, and the like, and is preferably made of a material with low cell adhesiveness. The gel used in the gel sheet may be one of those exemplified above as the gel plate medium and may be the same type or a different type of gel as in the gel plate medium.

### (Step b): Pipette Positioning Step)

Step b) is a step of positioning a nanopipette filled with a substance at a cell-corresponding position in the electrolytic solution. In this step, by freely selecting the penetration position of a nanopipette with a tip diameter of several tens of nanometers, a substance can be introduced into any single cell. Furthermore, the penetration site in a single cell can be freely selected, such as the cell nucleus or the cytoplasm. This method of site-selective substance introduction into a cell at the single cell level while being a minimally invasive method for cells is a unique feature of the present disclosure and cannot be achieved by existing methods. Methods of filling the inside of a nanopipette with a substance include, for example, a centrifugation method and a suction method. The centrifugation method may be performed by mounting the nanopipette in a centrifuge holder, filling the nanopipette with a substance through the base opening, and rotating the centrifuge holder with the nanopipette mounted thereon in a centrifuge. Suction may be accomplished by suctioning material from the tip opening. The nanopipette filled with the substance may be mounted, for example, in a three-dimensional (xyz) movement pipette holder as described above for positioning purposes. The “cell-corresponding position” refers to a position on the “z-axis” from the position of the target cell. Positioning may be accomplished with a rough actuator, a fine actuator, or a combination of both. The rough actuator and the fine actuator may be driven by manual control via a position input device or automatically controlled by a program. Furthermore, another method is to position the xy axes using a microscope stage.

### (Step c): Pipette Approach Step)

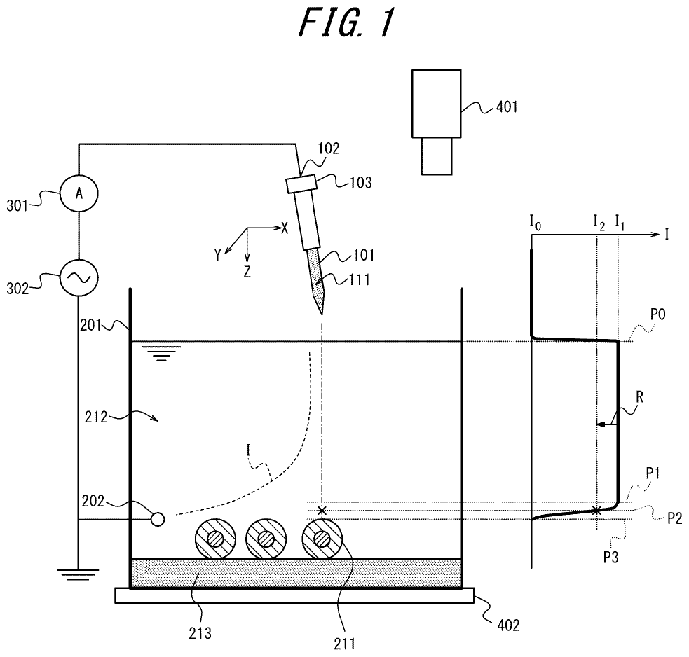

Step c) is a step of measuring the current between the inside of the nanopipette and the electrolytic solution and moving the nanopipette in the cell direction to a position at which the drop rate from the steady state current becomes the set current drop rate (the “pipette pause position (P2)” in FIG. 1). The “direction of approaching the cell” refers to the direction toward the cell on the “z-axis”. The current (ion current) between the inside of the nanopipette and the electrolytic solution can be measured by a current measurement circuit as the current between the pipette electrode and the reference electrode. In step b), in order to measure the current, it is preferable to apply a low voltage (set approach voltage) between the inside of the nanopipette and the electrolytic solution (i.e., between the pipette electrode and the reference electrode), the low voltage being set to a level that prevents the filling solution from flowing out due to electro-osmotic flow. The change in the current between the inside of the nanopipette and the electrolytic solution will be explained with reference to FIG. 1. When the tip of the nanopipette is located outside the electrolytic solution, the inside of the nanopipette is not connected to the electrolytic solution, and the current (I) becomes zero (I0). When the tip of the nanopipette reaches the electrolytic solution surface position (P0) and enters the electrolytic solution, the inside of the nanopipette and the electrolytic solution are connected, and the current (I) increases. For a while thereafter, even if the nanopipette is advanced, the current (I) remains steady without any significant change. The current (I) at this time is defined as a steady state current (I1). When the tip of the nanopipette reaches a position (“current drop starting point position (P1)”) extremely close to the cell surface position (P3), the current begins to drop rapidly according to the distance between the cell and the tip of the nanopipette. Thereafter, the nanopipette is advanced to a position (“pipette pause position (P2)”) at which the current (I) becomes a current (I2) that is reduced from the steady current (I1) by a set current drop rate (R). The nanopipette may be paused at the pipette pause position (P2). The movement of the nanopipette in step b) may be accomplished with a rough actuator, a fine actuator, or a combination of both. At least the movement after the current drop starting point position (P1) is preferably performed by a fine actuator. The movement of the nanopipette in step b) is preferably performed automatically by a program. By presetting the set current drop rate according to the cell type, it is possible to suppress damage to the cells and achieve highly efficient introduction of substances into the cells.

### <Set Current Drop Rate>

A value optimized for the cell type is used as the set current drop rate. The set current drop rate is preferably 2% to 50%, more preferably 10% to 40%, and most preferably 15% to 20%.

### <Set Approach Voltage>

A value optimized for the cell type is used as the set approach voltage. The value of the set approach voltage is preferably −2 V to +2 V, more preferably −1.0 V to +1.0 V.

### (Step d): Pipette Penetration Step)

Step d) is a step of moving the nanopipette from the aforementioned position (i.e., the pipette pause position (P2)) in the cell direction by a set penetration distance and penetrating the cell. “In the cell direction” refers to the direction towards the cell (the interior of the cell) on the “z-axis”. The movement of the nanopipette during the pipette penetration step is usually performed by a fine actuator under automatic control by a program. The nanopipette is preferably moved at a higher speed in the pipette penetration step than in the pipette approach step. By presetting the penetration distance according to the cell type, it is possible to suppress damage to the cells and achieve highly efficient introduction of substances into the cells.

### <Set Penetration Distance>

A value optimized for the cell type is used as the set penetration distance. The set penetration distance is preferably 1 μm to 50 μm, and more preferably 3 μm to 40 μm.

### (Step e): Substance Ejection Step)

Step e) is a step of applying a voltage between the inside of the nanopipette and the electrolytic solution to eject the substance into the cell. According to the type of substance being introduced into the cell (e.g., a protein, a mixture or complex containing a protein, a mixture or complex containing a protein and a nucleic acid, a nucleic acid, a dye, or the like), a voltage is applied so that the inside of the nanopipette is at a positive potential and the electrolytic solution is at a negative potential, or alternatively, so that the inside of the nanopipette is at a negative potential and the electrolytic solution is at a positive potential. When the substance to be introduced into the cell is a positively charged substance, a voltage is applied so that the inside of the nanopipette is at a positive potential and the electrolytic solution is at a negative potential. When the substance to be introduced into the cell is a negatively charged substance, a voltage is applied so that the inside of the nanopipette is at a negative potential and the electrolytic solution is at a positive potential. The voltage is preferably applied at a set injection voltage (set applied voltage: VI) and a set injection time (set application time: T1).

### <Set Injection Voltage>

A value optimized for the cell type is used as the set injection voltage. The set injection voltage is preferably −11 to +11 V, more preferably −10 to +10 V, and most preferably 4 V to 10 V, in the case of a positively charged substance. Furthermore, the set injection voltage can be optimized according to the cell type, as described in the “Examples of Setting Parameters” below.

### <Set Injection Time>

A value optimized for the cell type is used as the set injection time. The set injection time is preferably 0.1 s to 10 s, more preferably 0.5 s to 5.0 S.

### (Step f): Pipette Retraction Step)

Step f) is a step of retracting the nanopipette. The “direction away from the cell” refers to the direction away from the cell (interior of the cell) on the “z-axis”. The nanopipette movement may be performed at a set retraction distance. The movement of the nanopipette during the pipette retraction step is usually performed by a fine actuator under automatic control by a program.

### <Set Retraction Distance>

A value optimized for the cell type is used as the set retraction distance. The set retraction distance is preferably within a range of 40 μm or more.

### [Method of Producing a Genetically Modified Cell]

In one embodiment, the method of the present disclosure can also be used as a method of producing a genetically modified cell. The method of producing a genetically modified cell according to the present disclosure includes the following steps.
a) seeding floating cells on a gel plate medium of a specific hardness (floating cell seeding step);
b) filling the gel plate medium seeded with cells with an electrolytic solution and positioning a nanopipette filled with a substance at a cell-corresponding position in the electrolytic solution (pipette positioning step);
c) measuring a current between an inside of the nanopipette and the electrolytic solution and moving the nanopipette in a cell direction to a position at which a drop rate from a steady state current is a set current drop rate of 2% or more and 50% or less (pipette approach step);
d) moving the nanopipette in the cell direction by a set penetration distance of 1 μm or more and 50 μm or less to penetrate a cell (pipette penetration step);
e) applying a voltage between the inside of the nanopipette and the electrolytic solution to eject the substance into the cell (substance ejection step); and
f) retracting the nanopipette (pipette retraction step).

In another embodiment, a method of introducing a substance into a cell of the present disclosure includes the following steps.
a′) (floating cell gel sheet transfer step)
   a′-1) seeding floating cells onto a substrate and layering a gel on the cells;
   a′-2) peeling the gel from the substrate to obtain a gel sheet with the cells transferred to a gel surface;
   a′-3) layering the gel sheet on a gel plate medium so that a cell transfer surface faces up, and setting a hardness of layered gel overall, by a hardness evaluation according to JIS K6503, to a breaking load of 2.0 N to 30 N, Young's modulus of 0.5×10⁵ Pa to 7×10⁵ Pa, or 1 mm pressing stress of 1.0 N to 8 N;
b) filling the gel plate medium seeded with cells with an electrolytic solution and positioning a nanopipette filled with a substance at a cell-corresponding position in the electrolytic solution (pipette positioning step);
c) measuring a current between an inside of the nanopipette and the electrolytic solution and moving the nanopipette in a cell direction to a position at which a drop rate from a steady state current is a set current drop rate of 2% or more and 50% or less (pipette approach step);
d) moving the nanopipette in the cell direction by a set penetration distance of 1 μm or more and 50 μm or less to penetrate a cell (pipette penetration step);
e) applying a voltage between the inside of the nanopipette and the electrolytic solution to eject the substance into the cell (substance ejection step); and
f) retracting the nanopipette (pipette retraction step).

Genetic modification substances include positively charged genetic modification agents and negatively charged genetic modification agents. Examples of positively charged genetic modification substances include genetic modification substances based on mixtures or complexes containing proteins and nucleic acids, such as substances for genome editing (e.g., Cas9-sgRNA-RNP complexes used in the CRISPR-Cas9 system, as well as substances used in genome editing systems such as CRISPR-Cas3, ZFN, TALEN, and PPR). Examples of negatively charged genetic modification substances include nucleic acids (DNA, RNA) and the like. Examples of nucleic acids include DNA, such as plasmid vectors, and RNA, such as antisense RNA and siRNA.

The conditions for the method of producing a genetically modified cell can be the same as those described for the method of introducing a substance into a cell according to the present disclosure.

[Control Program for an Apparatus for Introducing a Substance into a Cell]

The present disclosure also provides a control program for an apparatus for introducing a substance into a cell. The program of the present disclosure includes instructions for performing the following steps.
a) positioning a nanopipette filled with a substance at a cell-corresponding position in an electrolytic solution in a gel plate medium of a specific hardness, the gel plate medium being seeded with floating cells and filled with the electrolytic solution (pipette positioning step);
b) measuring a current between an inside of the nanopipette and the electrolytic solution and moving the nanopipette in a cell direction to a position at which a drop rate from a steady state current is a set current drop rate of 2% or more and 50% or less (pipette approach step);
c) moving the nanopipette in the cell direction by a set penetration distance of 1 μm or more and 50 μm or less to penetrate a cell (pipette penetration step);
d) applying a voltage between the inside of the nanopipette and the electrolytic solution to eject the substance into the cell (substance ejection step); and
e) retracting the nanopipette (pipette retraction step).

The program of the present disclosure may further include instructions for controlling the approach voltage, the injection voltage (applied voltage), the injection time (application time), and the retraction distance.

The conditions controlled by the program of the present disclosure can be the same as those described for the method of introducing a substance into a cell according to the present disclosure.

### EXAMPLES

The present disclosure will be described in more detail below using examples, but the technical scope of the present disclosure is not limited to these examples.

### <Acquisition of Strain>

The microalgae Tetraselmis sp. and Haematococcus sp. used in this example were obtained as isolated products from the Microbial Culture Collection of the National Institute for Environmental Studies. Furthermore, Chlamydomonas reinhardtii Dangerd (ATCC PRA-142) was obtained from the American Type Culture Collection (ATCC) as a microalgae strain without a cell wall.

### <Preparation of Gel Plates for Freshwater Microalgae>

Gel plates for Haematococcus sp. and Chlamydomonas reinhardtii Dangerd (ATCC PRA-142) strains that mainly live in freshwater were prepared by the following procedure. Agarose (Agarose LM low melting point, manufactured by Nacalai) was added to BG11 medium (Thermo Fisher Scientific) to the concentrations listed in Table 1. Subsequently, sterilization was carried out using an autoclave at 121° C. for 20 minutes. Antibiotics (ampicillin and kanamycin at a final concentration of 50 μg/mL) were then added. Subsequently, about 5.8 mL was dispensed into a polystyrene petri dish, having a diameter of 3.5 cm and equipped with a position confirmation grid, and was allowed to stand to prepare a gel plate with a gel thickness of 6 mm.

### <Preparation of Gel Plate for Freshwater Microalgae Used in Gel Transfer Method>

In the preparation of the gel plates for freshwater microalgae described above, the amount of BG11 medium and a solution containing agarose and antibiotics dispensed into a polystyrene petri dish was approximately 4.3 mL in order to prepare a freshwater microalgae plate with a gel thickness of 4.5 mm for use in the gel transfer method.

### <Preparation of Gel Plate for Marine Microalgae>

Gel plates for a Tetraselmis sp. strain that mainly inhabits seawater were prepared by the following procedure. Agarose (Agarose LM low melting point, manufactured by Nacalai) was added to an IMK medium (manufactured by Fujifilm Wako Pure Chemical Corporation) to the concentrations listed in Table 1. Subsequently, sterilization was carried out using an autoclave at 121° C. for 20 minutes. Antibiotics (ampicillin and kanamycin at a final concentration of 50 μg/mL) were then added. Subsequently, about 5.8 mL was dispensed into a polystyrene petri dish, having a diameter of 3.5 cm and equipped with a position confirmation grid, and was allowed to stand to prepare a gel plate with a gel thickness of 6 mm.

### <Preparation of Gel Plate for Marine Microalgae Used in Gel Transfer Method>

In the preparation of the gel plate for marine microalgae described above, the amount of IMK medium and a solution containing agarose and antibiotics dispensed into a polystyrene petri dish was approximately 4.3 mL in order to prepare a marine microalgae plate with a gel thickness of 4.5 mm for use in the gel transfer method.

### <Inoculation of a Single Strain of Haematococcus sp.>

First, 15 μL of a microalgae suspension was dropped onto a gel plate for freshwater microalgae (gel thickness: 6 mm) and spread with a spreader. The result was incubated at 25° C. for several days to allow colonies to form. One colony was then collected using a sterile platinum loop and inoculated onto a fresh gel plate. The result was incubated at 25° C. for several days under a photon flux density of 25 to 100 μmol photons/m²/s to form colonies consisting of a single strain.

### <Inoculation of a Single Strain of Tetraselmis sp.>

First, 15 μL of a microalgae suspension was dropped onto a gel plate for marine microalgae (gel thickness: 6 mm) and spread with a spreader. The result was incubated at 25° C. for several days to allow colonies to form. One colony was then collected using a sterile platinum loop and inoculated onto a fresh gel plate IMK medium. The result was incubated at 25° C. for several days under a photon flux density of 25 to 100 μmol photons/m²/s to form colonies consisting of a single strain.

<Inoculation of a Single Strain of Chlamydomonas reinhardtii Dangerd (ATCC PRA-142)>

First, 15 μL of a microalgae suspension was dropped onto a gel plate for freshwater microalgae (gel thickness: 6 mm) and spread with a spreader. The result was incubated at 25° C. for several days to allow colonies to form. One colony was then collected using a sterile platinum loop and inoculated onto a fresh gel plate IMK medium. The result was incubated at 25° C. for several days under a photon flux density of 25 to 100 μmol photons/m²/s to form colonies consisting of a single strain.

### <Liquid Culture of a Single Strain of Haematococcus sp.>

First, 50 mL of sterilized BG11 liquid medium (manufactured by Thermo Fisher Scientific) was added to a sterilized 100 mL Erlenmeyer flask, and one colony was picked from the gel plate with a platinum loop and inoculated into the liquid medium. A sterilized silicone stopper was placed in the flask, and culturing with shaking was performed at 25° C. under a photon flux density of 25 to 100 μmol photons/m²/s for 3 days.

### <Liquid Culture of a Single Strain of Tetraselmis sp.>

First, 50 mL of sterilized IMK liquid medium (manufactured by Fujifilm Wako Pure Chemical Corporation) was added to a sterilized 50 mL Erlenmeyer flask, and one colony was picked from the gel plate with a platinum loop and inoculated into the liquid medium. A sterilized silicone stopper was placed in the flask, and culturing with shaking was performed at 25° C. under a photon flux density of 25 to 100 μmol photons/m²/s for 3 days.

<Liquid Culture of a Single Strain of Chlamydomonas reinhardtii Dangerd (ATCC PRA-142)>

First, 50 mL of sterilized IMK liquid medium (manufactured by Fujifilm Wako Pure Chemical Corporation) was added to a sterilized 50 mL Erlenmeyer flask, and one colony was picked from the gel plate with a platinum loop and inoculated into the liquid medium. A sterilized silicone stopper was placed in the flask, and culturing with shaking was performed at 25° C. under a photon flux density of 25 to 100 μmol photons/m²/s for 3 days.

<Preparation of Gel Plate with Haematococcus sp. Cells Fixed Thereon: One Gel Layer Configuration>

First, 2 mL of the microalgae cell suspension was collected, with a pipette or the like, from the Erlenmeyer flask in which the liquid culture had been carried out and was placed in a sterilized tube container. The concentration of the cell suspension was estimated using a counting chamber, and a cell suspension was obtained by adding BG11 liquid medium (manufactured by Thermo Fisher Scientific) to adjust the final concentration to 1.0×10⁵ cells/mL. Next, 15 μL of the cell suspension was dropped onto a gel plate for freshwater microalgae (gel thickness: 6 mm) on a 3.5 cm diameter petri dish prepared in advance, spread using a spreader, and left to stand for one day to obtain a gel plate with microalgae cells fixed thereon.

<Preparation of Gel Plate with Tetraselmis sp. Cells Fixed Thereon: One Gel Layer Configuration>

First, 2 mL of the microalgae cell suspension was collected, with a pipette or the like, from the Erlenmeyer flask in which the liquid culture had been carried out and was placed in a sterilized tube container. The concentration of the cell suspension was estimated using a counting chamber, and a cell suspension was obtained by adding IMK liquid medium (manufactured by Fujifilm Wako Pure Chemical Corporation) to adjust the final concentration to 1.0×10⁵ cells/mL. Next, 15 μL of the cell suspension was dropped onto a gel plate for marine microalgae (gel thickness: 6 mm) on a 3.5 cm diameter petri dish prepared in advance, spread using a spreader, and left to stand for one day to obtain a gel plate with microalgae cells fixed thereon.

<Preparation of Gel Plate with Chlamydomonas reinhardtii Dangerd (ATCC PRA-142) Cells Fixed Thereon: One Gel Layer Configuration>

First, 2 mL of the microalgae cell suspension was collected, with a pipette or the like, from the Erlenmeyer flask in which the liquid culture had been carried out and was placed in a sterilized tube container. The concentration of the cell suspension was estimated using a counting chamber, and a cell suspension was obtained by adding IMK liquid medium (manufactured by Fujifilm Wako Pure Chemical Corporation) to adjust the final concentration to 1.0×10⁵ cells/mL. Next, 15 μL of the cell suspension was dropped onto a gel plate for marine microalgae (gel thickness: 6 mm) on a 3.5 cm diameter petri dish prepared in advance, spread using a spreader, and left to stand for one day to obtain a gel plate with microalgae cells fixed thereon.

<Preparation of Gel Plate with Haematococcus sp. Cells Fixed Thereon by Gel Transfer Method: Two Gel Layer Configuration>

The fixation of cells by the gel transfer method for a Haematococcus sp. strain that mainly inhabits freshwater was carried out according to the following procedure. First, 2 mL of the microalgae cell suspension was collected, with a pipette or the like, from the Erlenmeyer flask in which the liquid culture had been carried out and was placed in a sterilized tube container. The concentration of the cell suspension was estimated using a counting chamber, and a cell suspension was obtained by adding BG11 liquid medium (manufactured by Thermo Fisher Scientific) to adjust the final concentration to 1.0×10⁵ cells/mL. Subsequently, 10 μL of the cell suspension was dropped onto an 18 mm×18 mm cover glass and allowed to spread under its own weight. Next, 200 μL of a solution containing agarose (Agarose LM low melting point, manufactured by Nacalai) in an amount yielding the concentrations listed in Table 1, and 50 μg/mL each of Ampicillin (manufactured by Fujifilm Wako Pure Chemical Corporation) and Kanamycin (manufactured by Fujifilm Wako Pure Chemical Corporation), in an IMK medium (manufactured by Fujifilm Wako Pure Chemical Corporation) was dropped onto the microalgae cells spread on the cover glass. A new 18 mm×18 mm cover glass was placed on top, sandwiching the microalgae cells and the gel-like medium containing agarose, and the result was allowed to stand at room temperature for 30 minutes. The cover glass on the side on which the microalgae cell suspension was dropped was then removed, and the microalgae cells were transferred to the gel sheet. Subsequently, the opposite cover glass was removed from the gel sheet to obtain a single layer of a gel sheet having a thickness of 1.5 mm and having the microalgae cells transferred onto its surface. The obtained single-layer gel sheet was layered on a gel plate for marine microalgae (gel thickness: 4.5 mm), used in the gel transfer method, on a previously prepared 3.5 cm diameter petri dish. The result was left to stand for 30 minutes to obtain a gel plate with microalgae cells fixed thereon by the gel transfer method.

<Preparation of Gel Plate with Tetraselmis sp. Cells Fixed Thereon by Gel Transfer Method: Two Gel Layer Configuration>

The fixation of cells by the gel transfer method for a Tetraselmis sp. strain that mainly inhabits seawater was carried out according to the following procedure. First, 2 mL of the microalgae cell suspension was collected, with a pipette or the like, from the Erlenmeyer flask in which the liquid culture had been carried out and was placed in a sterilized tube container. The concentration of the cell suspension was estimated using a counting chamber, and a cell suspension was obtained by adding IMK liquid medium (manufactured by Fujifilm Wako Pure Chemical Corporation) to adjust the final concentration to 1.0×10⁵ cells/mL. Subsequently, 10 μL of the cell suspension was dropped onto an 18 mm×18 mm cover glass and allowed to spread under its own weight. Next, 200 μL of a solution containing agarose (Agarose LM low melting point, manufactured by Nacalai) in an amount yielding the concentrations listed in Table 1, and 50 μg/mL each of Ampicillin (manufactured by Fujifilm Wako Pure Chemical Corporation) and Kanamycin (manufactured by Fujifilm Wako Pure Chemical Corporation), in an IMK medium (manufactured by Fujifilm Wako Pure Chemical Corporation) was dropped onto the microalgae cells spread on the cover glass. A new 18 mm×18 mm cover glass was placed on top, sandwiching the microalgae cells and the gel-like medium containing agarose, and the result was allowed to stand at room temperature for 30 minutes. The cover glass on the side on which the microalgae cell suspension was dropped was then removed, and the microalgae cells were transferred to the gel sheet. Subsequently, the opposite cover glass was removed from the gel sheet to obtain a single layer of a gel sheet having a thickness of 1.5 mm and having the microalgae cells transferred onto its surface. The obtained single-layer gel sheet was layered on a gel plate for marine microalgae (gel thickness: 4.5 mm), used in the gel transfer method, on a previously prepared 3.5 cm diameter petri dish. The result was left to stand for 30 minutes to obtain a gel plate with microalgae cells fixed thereon by the gel transfer method.

### <Preparation of Reagent Solution>

FITC-dextran (manufactured by Merck) was diluted with PBS to a concentration of 10 mg/mL to obtain a FITC dextran/PBS solution. Furthermore, GFP (Merck) was diluted with PBS to a concentration of 0.4 mg/mL to obtain a GFP/PBS suspension. Furthermore, FAM-Oligo DNA (90 mer, 29.7 kDa, manufactured by Eurofins Genomics) was diluted with PBS to a concentration of 100 μM to obtain a FAM-Oligo DNA/PBS suspension.

<Preparation of Nanopipette Filled with Reagent Solution>

First, 5 μL of the FITC-dextran/PBS solution, GFP/PBS suspension, or FAM-Oligo DNA suspension was placed in a micro loader attached to a centrifuge holder, loaded into the nanopipette from the top, and rotated for 30 s to 60 s in a tabletop centrifuge. The silver wire attached to the nanopipette was inserted through the hole at the top filled with the reagent solution and fixed with a special jig. At this time, it was confirmed that the silver wire was immersed in the reagent solution.

<Injection of Reagent Solution into Microalgae Cells>

PBS as an electrolytic solution was poured in an amount of 3 mL onto the gel plate medium (a petri dish with a diameter of 3.5 cm) on which the microalgae cells were fixed. The gel plate sample on which the microalgae cells were fixed was set on a microscope (Ti2E, manufactured by Nikon). The nanopipette filled with the reagent solution was attached to the head of an apparatus for introducing a substance into microalgae cells (Single Cellome™ System UNIT SU10, manufactured by Yokogawa), and the rotary angle was adjusted. The reference electrode attached to the SU10 head was placed in the PBS electrolytic solution of the gel plate sample on which the microalgae was fixed, and the SU10 software (measurement mode) was started. The nanopipette was brought into contact with PBS, which is an electrolytic solution, either manually or using the Liquid detect mode, and it was confirmed by software that the current value had increased from 0 nA. The SU10 joystick was manipulated to position the nanopipette at a position corresponding to a target cell in the sample. The SU10 software was switched from measurement mode to delivery mode. Parameters were set according to the microalgae samples listed in Table 1. In the SU10 software, start was pressed, the current between the inside of the nanopipette and the electrolytic solution was measured, the nanopipette was moved in the cell direction to a position at which the drop rate from a steady state current was a set current drop rate of 2.5% or more and 49.5% or less, and the nanopipette was then moved in the cell direction by a set penetration distance of 1.5 μm or more and 49.5 μm or less to perform a reagent solution substance introduction process into the target cell. Next, the joystick was operated to position the nanopipette at a position corresponding to the next target cell. The substance was introduced into 40 cells on the gel plate sample per condition.

### <Method of Evaluating Gel Plate Hardness>

The hardness of the gel plate was measured using a rheometer under conditions conforming to the jelly strength measurement specified in JIS K6503. Specifically, the gel plate was placed in a rheometer (CR-100, manufactured by Sun Scientific), and a plunger (diameter 12.7 mm, height 35 mm) was inserted into the gel plate at a speed of 1 mm/s. Young's modulus [Pa], breaking load [N], and stress at 1 mm insertion [N] at this time were evaluated as indicators of the gel plate hardness.

### <Method for Evaluating Success Rate of Substance Introduction>

The efficiency of substance introduction into microalgae cells was evaluated based on how many of the 40 cells into which the substance was introduced exhibited fluorescence emission derived from the introduced substance. Specifically, the gel plate sample was set on a microscope (Ti2E, manufactured by Nikon), and the number of cells in which FITC-dextran-derived fluorescence emission at around 518 nm (excitation wavelength 475 nm) or GFP-derived fluorescence emission at around 507 nm (excitation wavelength 475 nm) was observed in the fluorescence observation mode was counted, and the substance introduction efficiency (success rate of substance introduction) was calculated by the formula “number of cells with observed fluorescence emission/40 cells×100”. The evaluation indicators for the substance introduction efficiency are as follows.
0% or more but less than 2%: poor
2% or more but less than 10%: fair
10% or more but less than 30%: good
30% or more and 100% or less: excellent

### <Method of Evaluating Maintenance of Cell Division Function>

The cell survival rate was evaluated by statically culturing the cells into which the substance was introduced on a gel plate at 25° C. for 3 days under a photon flux density of 25 to 100 μmol photons/m²/s, and then observing the cells under a microscope to evaluate the presence of divided cells resulting from cell division, using the following indicators.
Out of 40 cells, 4 or fewer cells have undergone cell division: poor
Out of 40 cells, 5 or more cells but 29 or fewer cells have undergone cell division: fair
Out of 40 cells, 30 or more cells have undergone cell division: good

As illustrated by the examples, when floating cells are fixed to a gel plate of a specific hardness and combined with specific penetration conditions using a nanopipette, the effect of achieving both high efficiency of substance introduction into floating cells and a high cell survival rate is achieved. By fixing floating cells on a gel plate of a specific hardness, a substance can be introduced in particular into cells that have a hard cell wall, without the cells moving when contacted with the nanopipette. Furthermore, the method of layering a gel sheet with cells transferred to the gel onto a gel plate tended to inhibit cell migration better and resulted in a higher success rate of substance introduction.

Furthermore, the comparative examples demonstrate that simply setting the gel plate hardness within a specific range to inhibit cell migration when penetrating with a nanopipette is insufficient, since even if the substance is successfully introduced into cells with a cell wall, reduced cell survival after penetration may be observed. According to the present disclosure, the survival rate of cells can be improved by using a specific gel plate hardness and specific nanopipette penetration conditions. This is thought to be because, by using a gel plate of a specific hardness and specific nanopipette penetration conditions, the cells are appropriately fixed to the gel plate medium, the restoring force of the gel plate medium and the penetration pressure of the nanopipette device minimize the physical impact on the cells, and the weak ionic current reduces the cell load. Furthermore, it is thought that the weak current that flows through the cell when penetrated at the hardness of the medium of the present disclosure contributes to repairing cell damage.

TABLE 1

| | Gel plate medium hardness | | | | Agarose | | Nanopipette conditions | |
|---|---|---|---|---|---|---|---|---|
| | Young's | Breaking | Stress at 1 mm | | concentration | | Injection | Injection |
| Sample | modulus | load | insertion | Gel layer | (gel plate medium/ | | voltage | time |
| No. | [Pa] | [N] | [N] | composition | transfer gel sheet) | | [V] | [s] |
| 1 | 0.75 × 10\hat{ }5 | 2.2 | 1.3 | 1 layer | 0.5 | w % | 6 | 1 |
| 2 | 1.0 × 10\hat{ }5 | 2.7 | 1.9 | 1 layer | 0.7 | w % | 6 | 1 |
| 3 | 2.5 × 10\hat{ }5 | 9.7 | 4.1 | 1 layer | 1.5 | w % | 6 | 1 |
| 4 | 5.6 × 10\hat{ }5 | 27 | 7.2 | 1 layer | 3.0 | w % | 6 | 1 |
| 5 | 0.75 × 10\hat{ }5 | 2.2 | 1.3 | 1 layer | 0.5 | w % | 6 | 1 |
| 6 | 2.5 × 10\hat{ }5 | 9.7 | 4.1 | 1 layer | 1.5 | w % | 2 | 0.5 |
| 7 | 2.5 × 10\hat{ }5 | 9.7 | 4.1 | 1 layer | 1.5 | w % | 4 | 0.5 |
| 8 | 2.5 × 10\hat{ }5 | 9.7 | 4.1 | 1 layer | 1.5 | w % | 6 | 0.5 |
| 9 | 2.3 × 10\hat{ }5 | 9.4 | 4 | 2 layers | 1.5 w %/1.5 w % | | 2 | 0.5 |
| 10 | 2.3 × 10\hat{ }5 | 9.4 | 4 | 2 layers | 1.5 w %/1.5 w % | | 4 | 0.5 |
| 11 | 2.3 × 10\hat{ }5 | 9.4 | 4 | 2 layers | 1.5 w %/1.5 w % | | 5 | 0.5 |
| 12 | 2.3 × 10\hat{ }5 | 9.4 | 4 | 2 layers | 1.5 w %/1.5 w % | | 6 | 0.5 |
| 13 | 2.3 × 10\hat{ }5 | 9.4 | 4 | 2 layers | 1.5 w %/1.5 w % | | 8 | 0.5 |
| 14 | 2.3 × 10\hat{ }5 | 9.4 | 4 | 2 layers | 1.5 w %/1.5 w % | | 10 | 0.5 |
| 15 | 2.3 × 10\hat{ }5 | 9.4 | 4 | 2 layers | 1.5 w %/1.5 w % | | 5 | 2 |
| 16 | 2.3 × 10\hat{ }5 | 9.4 | 4 | 2 layers | 1.5 w %/1.5 w % | | 5 | 1 |
| 17 | 2.3 × 10\hat{ }5 | 9.4 | 4 | 2 layers | 1.5 w %/1.5 w % | | 5 | 0.1 |
| 18 | 2.3 × 10\hat{ }5 | 9.4 | 4 | 2 layers | 1.5 w %/1.5 w % | | 5 | 0.5 |
| 19 | 4.3 × 10\hat{ }5 | 22 | 5.3 | 1 layer | 2 | w % | 3 | 0.1 |
| 20 | 2.3 × 10\hat{ }5 | 9 | 4 | 2 layers | 2 | w % | −5 | 0.5 |
| 21 | 2.3 × 10\hat{ }5 | 9 | 4 | 2 layers | 2 | w % | −7 | 0.5 |
| 22 | 0.4 × 10\hat{ }5 | 1.2 | 0.7 | 1 layer | 0.3 | w % | 6 | 1 |
| 23 | 8.1 × 10\hat{ }5 | 37.5 | 10 | 1 layer | 5 | w % | 6 | 1 |
| 24 | 8.0 × 10\hat{ }5 | 36.5 | 9.5 | 2 layers | 5 w %/5 w % | | 6 | 1 |
| 25 | 2.5 × 10\hat{ }5 | 9.7 | 4.1 | 1 layer | 1.5 | w % | 5 | 0.05 |
| 26 | 2.5 × 10\hat{ }5 | 9.7 | 4.1 | 1 layer | 1.5 | w % | 5 | 0.01 |
| 27 | 2.5 × 10\hat{ }5 | 9.7 | 4.1 | 1 layer | 1.5 | w % | 6 | 10 |

| | | | Evaluation | | |
|---|---|---|---|---|---|
| | | | Success rate | | |
| Sample | Introduced | Microalgae | of substance | Cell | |
| No. | substance | species | introduction | division | Notes |
| 1 | FITC-dextran | Haematococcus sp. | Excellent: 40% | — | Example 1 |
| 2 | FITC-dextran | Haematococcus sp. | Excellent: 50% | — | Example 2 |
| 3 | FITC-dextran | Haematococcus sp. | Excellent: 44% | — | Example 3 |
| 4 | FITC-dextran | Haematococcus sp. | Excellent: 32% | — | Example 4 |
| 5 | FITC-dextran | Tetraselmis sp. | Good: 10% | Good | Example 5 |
| 6 | FITC-dextran | Tetraselmis sp. | Good: 20% | Good | Example 6 |
| 7 | FITC-dextran | Tetraselmis sp. | Good: 13% | Good | Example 7 |
| 8 | FITC-dextran | Tetraselmis sp. | Good: 13% | Good | Example 8 |
| 9 | FITC-dextran | Tetraselmis sp. | Fair: 3% | Good | Example 9 |
| 10 | FITC-dextran | Tetraselmis sp. | Good: 23% | Good | Example 10 |
| 11 | FITC-dextran | Tetraselmis sp. | Excellent: 45% | Good | Example 11 |
| 12 | FITC-dextran | Tetraselmis sp. | Good: 23% | Good | Example 12 |
| 13 | FITC-dextran | Tetraselmis sp. | Good: 25% | Good | Example 13 |
| 14 | FITC-dextran | Tetraselmis sp. | Good: 20% | Good | Example 14 |
| 15 | FITC-dextran | Tetraselmis sp. | Good: 23% | Good | Example 15 |
| 16 | FITC-dextran | Tetraselmis sp. | Excellent: 30% | Good | Example 16 |
| 17 | FITC-dextran | Tetraselmis sp. | Good: 13% | Good | Example 17 |
| 18 | GFP | Tetraselmis sp. | Good: 10% | Good | Example 18 |
| 19 | FITC-dextran | Chlamydomonas | Fair: 7% | — | Example 19 |
| | | reinhardtii Dangeard | | | |
| | | (ATCC PRA-142) | | | |
| | | (no cell wall) | | | |
| 20 | FAM-Oligo DNA | Tetraselmis sp. | Good: 28% | Good | Example 20 |
| 21 | FAM-Oligo DNA | Tetraselmis sp. | Good: 20% | Good | Example 21 |
| 22 | FITC-dextran | Haematococcus sp. | Poor: 0% | — | Comparative Example 1 |
| 23 | FITC-dextran | Haematococcus sp. | Good: 11% | Poor | Comparative Example 2 |
| 24 | FITC-dextran | Haematococcus sp. | Good: 25% | Poor | Comparative Example 3 |
| 25 | FITC-dextran | Tetraselmis sp. | Poor: 0% | — | Comparative Example 4 |
| 26 | FITC-dextran | Tetraselmis sp. | Poor: 0% | — | Comparative Example 5 |
| 27 | FITC-dextran | Tetraselmis sp. | Good: 18% | Poor | Comparative Example 6 |

### INDUSTRIAL APPLICABILITY

According to the present disclosure, it is possible to provide a method of introducing a substance into a cell, the method achieving both high substance introduction efficiency and a high cell survival rate. Furthermore, using this method of introducing a substance, the present disclosure can provide an automatically controlled system for introducing a substance into a cell. This technique makes it possible to introduce substances such as nucleic acids and genome editing tools in particular into cells with a cell wall, thereby contributing to improved breeding efficiency.

1. A method of introducing a substance into a cell, the method comprising:
a) seeding floating cells on a gel plate medium having a hardness such that, by a hardness evaluation according to JIS K6503, a breaking load is 2.0 N to 30 N, Young's modulus is 0.5×10⁵ Pa to 7×10⁵ Pa, or 1 mm pressing stress is 1.0 N to 8 N;
b) filling the gel plate medium seeded with cells with an electrolytic solution and positioning a nanopipette filled with a substance at a cell-corresponding position in the electrolytic solution;
c) measuring a current between an inside of the nanopipette and the electrolytic solution and moving the nanopipette in a cell direction to a position at which a drop rate from a steady state current is a set current drop rate of 2% or more and 50% or less;
d) moving the nanopipette in the cell direction by a set penetration distance of 1 μm or more and 50 μm or less to penetrate a cell;
e) applying a voltage between the inside of the nanopipette and the electrolytic solution to eject the substance into the cell; and
f) retracting the nanopipette.

2. A method of introducing a substance into a cell, the method comprising:
a′)
a′-1) seeding floating cells onto a substrate and layering a gel on the cells;
a′-2) peeling the gel from the substrate to obtain a gel sheet with the cells transferred to a gel surface;
a′-3) layering the gel sheet on a gel plate medium so that a cell transfer surface faces up, and setting a hardness of layered gel overall, by a hardness evaluation according to JIS K6503, to a breaking load of 2.0 N to 30 N, Young's modulus of 0.5×10⁵ Pa to 7×10⁵ Pa, or 1 mm pressing stress of 1.0 N to 8 N;
b) filling the gel plate medium seeded with cells with an electrolytic solution and positioning a nanopipette filled with a substance at a cell-corresponding position in the electrolytic solution;
c) measuring a current between an inside of the nanopipette and the electrolytic solution and moving the nanopipette in a cell direction to a position at which a drop rate from a steady state current is a set current drop rate of 2% or more and 50% or less;
d) moving the nanopipette in the cell direction by a set penetration distance of 1 μm or more and 50 μm or less to penetrate a cell;
e) applying a voltage between the inside of the nanopipette and the electrolytic solution to eject the substance into the cell; and
f) retracting the nanopipette.

3. The method according to claim 1, wherein the substance is a protein, a mixture or complex containing a protein, a mixture or complex containing a protein and a nucleic acid, a nucleic acid, or a dye.

4. The method according to claim 3, wherein the substance is a substance for genome editing.

5. The method according to claim 3, wherein
the substance is a positively charged substance or a negatively charged substance,
in a case in which the substance is a positively charged substance, e) is performed by applying a voltage so that the inside of the nanopipette is at a positive potential and the electrolytic solution is at a negative potential, and
in a case in which the substance is a negatively charged substance, e) is performed by applying a voltage so that the inside of the nanopipette is at a negative potential and the electrolytic solution is at a positive potential.

6. The method according to claim 5, wherein e) is performed by applying a voltage at a set applied voltage of −11 V or more and +11 V or less and a set application time of 0.1 seconds or more and 5.0 seconds or less.

7. A method of producing a genetically modified cell, the method comprising:
a) seeding floating cells on a gel plate medium having a hardness such that, by a hardness evaluation according to JIS K6503, a breaking load is 2.0 N to 30 N, Young's modulus is 0.5×10⁵ Pa to 7×10⁵ Pa, or 1 mm pressing stress is 1.0 N to 8 N;
b) filling the gel plate medium seeded with cells with an electrolytic solution and positioning a nanopipette filled with a substance at a cell-corresponding position in the electrolytic solution;
c) measuring a current between an inside of the nanopipette and the electrolytic solution and moving the nanopipette in a cell direction to a position at which a drop rate from a steady state current is a set current drop rate of 2% or more and 50% or less;
d) moving the nanopipette in the cell direction by a set penetration distance of 1 μm or more and 50 μm or less to penetrate a cell;
e) applying a voltage between the inside of the nanopipette and the electrolytic solution to eject the substance into the cell; and
f) retracting the nanopipette.

8. A method of producing a genetically modified cell, the method comprising:
a′)
a′-1) seeding floating cells onto a substrate and layering a gel on the cells;
a′-2) peeling the gel from the substrate to obtain a gel sheet with the cells transferred to a gel surface;
a′-3) layering the gel sheet on a gel plate medium so that a cell transfer surface faces up, and setting a hardness of layered gel overall, by a hardness evaluation according to JIS K6503, to a breaking load of 2.0 N to 30 N, Young's modulus of 0.5×10⁵ Pa to 7×10⁵ Pa, or 1 mm pressing stress of 1.0 N to 8 N;
b) filling the gel plate medium seeded with cells with an electrolytic solution and positioning a nanopipette filled with a substance at a cell-corresponding position in the electrolytic solution;
c) measuring a current between an inside of the nanopipette and the electrolytic solution and moving the nanopipette in a cell direction to a position at which a drop rate from a steady state current is a set current drop rate of 2% or more and 50% or less;
d) moving the nanopipette in the cell direction by a set penetration distance of 1 μm or more and 50 μm or less to penetrate a cell;
e) applying a voltage between the inside of the nanopipette and the electrolytic solution to eject the substance into the cell; and
f) retracting the nanopipette.

9. A genetically modified cell produced by a method comprising:
a) seeding floating cells on a gel plate medium having a hardness such that, by a hardness evaluation according to JIS K6503, a breaking load is 2.0 N to 30 N, Young's modulus is 0.5×10⁵ Pa to 7×10⁵ Pa, or 1 mm pressing stress is 1.0 N to 8 N;
b) filling the gel plate medium seeded with cells with an electrolytic solution and positioning a nanopipette filled with a substance at a cell-corresponding position in the electrolytic solution;
c) measuring a current between an inside of the nanopipette and the electrolytic solution and moving the nanopipette in a cell direction to a position at which a drop rate from a steady state current is a set current drop rate of 2% or more and 50% or less;
d) moving the nanopipette in the cell direction by a set penetration distance of 1 μm or more and 50 μm or less to penetrate a cell;
e) applying a voltage between the inside of the nanopipette and the electrolytic solution to eject the substance into the cell; and
f) retracting the nanopipette.

10. A genetically modified cell produced by a method comprising:
a′)
a′-1) seeding floating cells onto a substrate and layering a gel on the cells;
a′-2) peeling the gel from the substrate to obtain a gel sheet with the cells transferred to a gel surface;
a′-3) layering the gel sheet on a gel plate medium so that a cell transfer surface faces up, and setting a hardness of layered gel overall, by a hardness evaluation according to JIS K6503, to a breaking load of 2.0 N to 30 N, Young's modulus of 0.5×10⁵ Pa to 7×10⁵ Pa, or 1 mm pressing stress of 1.0 N to 8 N;
b) filling the gel plate medium seeded with cells with an electrolytic solution and positioning a nanopipette filled with a substance at a cell-corresponding position in the electrolytic solution;
c) measuring a current between an inside of the nanopipette and the electrolytic solution and moving the nanopipette in a cell direction to a position at which a drop rate from a steady state current is a set current drop rate of 2% or more and 50% or less;
d) moving the nanopipette in the cell direction by a set penetration distance of 1 μm or more and 50 μm or less to penetrate a cell;
e) applying a voltage between the inside of the nanopipette and the electrolytic solution to eject the substance into the cell; and
f) retracting the nanopipette.

11. A control program for an apparatus for introducing a substance into a cell, the control program comprising instructions for executing processing comprising:
a) positioning a nanopipette filled with a substance at a cell-corresponding position in an electrolytic solution in a gel plate medium of a specific hardness, the gel plate medium being seeded with floating cells and filled with the electrolytic solution;
b) measuring a current between an inside of the nanopipette and the electrolytic solution and moving the nanopipette in a cell direction to a position at which a drop rate from a steady state current is a set current drop rate of 2% or more and 50% or less;
c) moving the nanopipette in the cell direction by a set penetration distance of 1 μm or more and 50 μm or less to penetrate a cell;
d) applying a voltage between the inside of the nanopipette and the electrolytic solution to eject the substance into the cell; and
e) retracting the nanopipette.
